(19) United States
(12) Patent Application Publication
(10) Pub. No.: US 2019/0231322 A1
(43) Pub. Date: Aug. 1, 2019

(54) CONTAINER TO SUPPORT TISSUE SAMPLE TRAY

(71) Applicants: Devicor Medical Products, Inc., Cincinnati, OH (US); Leica Microsystems Ltd., Shanghai (CN)

(72) Inventors: Bryan Keller, Cincinnati, OH (US); Joanne Fleming, Boulder, CO (US); Chonglu Wang, Shanghai (CN)

(21) Appl. No.: 16/380,359

(22) Filed: Apr. 10, 2019

Related U.S. Application Data
(63) Continuation of application No. PCT/CN2016/101816, filed on Oct. 11, 2016.

(51) Int. Cl. A61B 10/00; A61B 10/02

(52) U.S. Cl. CPC A61B 10/0096; A61B 10/0283; A61B 2010/0225; A61B 10/0275

(57) ABSTRACT

A container contains a tissue sample tray with tissue samples disposed thereon. The container fits in a radiograph machine and thereby allows the tissue samples to be radiographically imaged. The container also defines openings allowing fixation fluid to immerse the tissue samples in the container when the container is immersed in the fixation fluid. The container includes a base and a lid. The base includes a floor and sidewalls that cooperate to define a compartment, which is configured to receive a tissue sample tray. A plurality of bosses extend upwardly from the floor and are configured to maintain positioning of a tissue sample tray received in the compartment. The lid is configured to enable placement of a tissue sample tray in the compartment when the lid is open. The lid is configured to enclose a tissue sample tray in the compartment when the lid is closed.

### FIELD

This invention is in the field of obtaining tissue samples during biopsy procedures and then analyzing these tissue samples.

### BACKGROUND

A biopsy is the removal of a tissue sample from a patient to enable examination of the tissue for signs of cancer or other disorders. Tissue samples may be obtained in a variety of ways using various medical procedures involving a variety of the sample collection devices. For example, biopsies may be open procedures (surgically removing tissue after creating an incision) or percutaneous procedures (e.g. by fine needle aspiration, core needle biopsy, or vacuum assisted biopsy). After the tissue sample is collected, the tissue sample may be analyzed at a lab (e.g. a pathology lab, biomedical lab, etc.) that is set up to perform the appropriate tests (such as histological analysis).

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

The state of the art for breast biopsy is vacuum-assisted breast biopsy. A current textbook in this area is “Vacuum-Assisted Breast Biopsy with Mammotome®” available Nov. 11, 2012, copyright 2013 by Devicor Medical Germany GmBh, published in Germany by Springer Medizin Verlag, Authors: Markus Hahn, Anne Tardivon and Jan Casselman, ISBN

Biopsy devices may be used under ultrasound image guidance, stereotactic (X-ray) guidance, MRI guidance, Positron Emission Mammography (“PEM” guidance), Breast-Specific Gamma Imaging (“BSGI”) guidance, or otherwise. Each procedure has its own methodology based on the form of imaging guidance used. The following briefly describes ultrasound image guided biopsy procedures, stereotactic guided biopsy procedures and MRI guided biopsy procedures.

In an ultrasound image guided breast biopsy procedure, the operator may position an ultrasound transducer on the patient's breast and maneuver the transducer while viewing an ultrasound image display screen to locate suspicious tissue in the patient's breast. Once the operator locates the suspicious tissue, the operator may anesthetize the target region of the breast. Once the breast has been anesthetized, the operator may create an initial incision using a scalpel at a location on the exterior of the breast offset from the transducer. A needle of a breast biopsy probe disposed coaxially within an introducer cannula is then inserted into the breast through the initial incision. The operator continues to hold the ultrasound transducer with one hand while maneuvering the biopsy probe with the other hand. While viewing the ultrasound image on the display screen, the operator guides the needle to a position adjacent to the suspicious tissue. A cutter within the needle of the probe is used to remove tissue which is then conveyed either to a manual pick-up location on the breast biopsy device or to a tissue sample chamber. The needle of the breast biopsy device is then removed, leaving the introducer cannula disposed within the breast. The introducer cannula may then be used to introduce a biopsy marker cannula for deploying a biopsy site marker at the biopsy site. Once a marker has been deployed at the biopsy site, the biopsy marker cannula and the introducer cannula are both removed from the breast and the incision is closed using a medically acceptable way to close breaks in the skin.

In a stereotactic image guided breast biopsy procedure, the patient is first positioned relative to x-ray equipment, which includes a breast localization assembly. In some procedures, the patient is oriented in a prone position, with the patient lying face down on a procedure table with at least one breast hanging pendulously through an aperture in the procedure table. The breast is then compressed between a compression paddle and an x-ray receptor of a localization assembly that is positioned under the procedure table. A breast biopsy device is positioned on an automatic guide device in front of the compression paddle and between the breast and an x-ray source. Once positioning of the patient and localization of the breast are complete, a scout image is acquired with the x-ray receptor in a zero-degree angular position (i.e., the x-rays are emitted along an axis normal relative to the x-ray receptor). If the scout image indicates that the patient has been positioned in a desired position, the procedure may proceed with the acquisition of stereotactic image pairs. Stereotactic image pairs are acquired by orienting the x-ray source at various complementary angular positions relative to the x-ray receptor (e.g., +15° and)−15°, with at least one x-ray image acquired at each position.

Further in the stereotactic image guided breast biopsy procedure, once a suitable stereotactic image pair is acquired, an operator may identify a target site where biopsy sampling is desired by examining the stereotactic image pair. The target site is marked on each stereotactic image and a precise location of the target site on a Cartesian coordinate system is computed using an image processing module. The computed location of the target site is then communicated to the automatic guide device. The automatic guide device is responsive to this information to position the breast biopsy probe into a position that aligns with the target site. With the breast biopsy device positioned, an operator may then fire a needle of the biopsy probe into the breast of the patient, thereby positioning the needle at the target site. A cutter within the needle of the probe is used to remove tissue, which is then conveyed either to a manual pick-up location on the breast biopsy device or to a tissue sample chamber. After the biopsy tissue is removed, a biopsy marker cannula is inserted into the needle and is used to deploy a biopsy site marker at the biopsy site. Once a marker has been deployed at the biopsy site, the needle is removed from the breast and the incision is closed using a medically acceptable way to close breaks in the skin.

In an MRI guided breast biopsy procedure, after the patient is properly positioned on the table and a targeting device (e.g., a grid and cube combination or a pillar, post and cradle support combination) has been deployed and used, a baseline MRI image is taken to verify the target location. After that, a scalpel is used to incise the skin of the breast. Next, an assembly, formed by an obturator disposed in a sleeve, is inserted through the incision to penetrate the breast tissue under the skin. In some acceptable surgical techniques, the obturator is removed and an imaging rod is inserted into the sleeve in place of the obturator. An imaging rod is defined simply as an appropriately shaped rod that includes a feature that is detectable by an imaging technique being used for the biopsy procedure. The MRI image of the imaging rod is used to locate the site to which the sleeve/obturator assembly has penetrated. In some other acceptable surgical techniques, the obturator cooperates with the breast tissue to provide a visually observable artifact in an MRI image. With both of these techniques, after the location within the breast where the biopsy is to be taken is confirmed, the obturator or the imaging rod is removed.

Further in the MRI guided breast biopsy procedure, after the obturator or imaging rod has been removed, it is replaced in the sleeve with the needle of a breast biopsy probe. A cutter within the needle of the probe is used to remove tissue, which is then conveyed either to a manual pick up location on the breast biopsy device or to a breast biopsy device sample chamber. After the biopsy tissue is removed, a biopsy marker cannula is inserted into the needle and is used to deploy a biopsy site marker at the biopsy site. The needle is then removed from the sleeve. Optionally, the imaging rod or the obturator is put back into the breast for reimaging of the biopsy site. Then the imaging rod or obturator and the sleeve are removed.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “MRI Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 1, 2012; U.S. Pat. No. 8,206,316, entitled “Tetherless Biopsy Device with Reusable Portion,” issued on Jun. 26, 2012; U.S. Pat. No. 8,241,226, entitled “Biopsy Device with Rotatable Tissue Sample Holder,” issued on Aug. 14, 2012; U.S. Pat. No. 8,251,916, entitled “Revolving Tissue Sample Holder for Biopsy Device,” issued Aug. 28, 2012; U.S. Pat. No. 8,454,531, entitled “Icon-Based User Interface on Biopsy System Control Module,” published May 21, 2009, issued on Jun. 4, 2013; U.S. Pat. No. 8,532,747, entitled “Biopsy Marker Delivery Device,” issued Sep. 10, 2013; U.S. Pat. No. 8,702,623, entitled “Biopsy Device with Discrete Tissue Chambers,” issued on Apr. 22, 2014; U.S. Pat. No. 8,764,680, entitled “Handheld Biopsy Device with Needle Firing,” issued on Jun. 11, 2014; U.S. Pat. No. 8,801,742, entitled “Needle Assembly and Blade Assembly for Biopsy Device,” issued Aug. 12, 2014; U.S. Pat. No. 8,858,465, entitled “Biopsy Device with Motorized Needle Firing,” issued Oct. 14, 2014; U.S. Pat. No. 8,938,285, entitled “Access Chamber and Markers for Biopsy Device,” issued Jan. 20, 2015; U.S. Pat. No. 9,095,326, entitled “Biopsy System with Vacuum Control Module,” issued Aug. 4, 2015; U.S. Pat. No. 9,095,326, entitled “Biopsy System with Vacuum Control Module,” issued Aug. 4, 2015 and U.S. Patent No. 9,326,755, entitled “Biopsy Device Tissue Sample Holder with Bulk Chamber and Pathology Chamber,” issued May 3, 2016. The disclosure of each of the above-cited U.S. Patents is incorporated by reference herein.

Additional exemplary biopsy devices and biopsy system components are disclosed in U.S. Pub. No. 2006/0074345, entitled “Biopsy Apparatus and Method,” published Apr. 6, 2006 and now abandoned; U.S. Pub. No. 2008/0214955, entitled “Presentation of Biopsy Sample by Biopsy Device,” published Sep. 4, 2008; U.S. Pub. No. 2009/0131821, entitled “Graphical User Interface For Biopsy System Control Module,” published May 21, 2009, now abandoned; U.S. Pub. No. 2010/0152610, entitled “Hand Actuated Tetherless Biopsy Device with Pistol Grip,” published Jun. 17, 2010, now abandoned; U.S. Pub. No. 2010/0160819, entitled “Biopsy Device with Central Thumbwheel,” published Jun. 24, 2010, now abandoned; U.S. Pub. No. 2013/0144188, entitled “Biopsy Device With Slide-In Probe,” published Jun. 6, 2013; and U.S. Pub. No. 2013/0324882, entitled “Control for Biopsy Device,” published Dec. 5, 2013. The disclosure of each of the above-cited U.S. Patent Application Publications is incorporated by reference herein.

U.S. Pub. No. 2014/0275999, entitled “Biopsy device” published Sep. 18, 2014, and U.S. Pub. No. 2016/0183928, entitled “Biopsy Device,” published Jun. 30, 2016, both describe some aspect of a biopsy device including a probe, a holster, and a tissue sample holder for collecting tissue samples. The probe includes a needle and a hollow cutter. The tissue sample holder includes a housing having a plurality of chambers that are configured to receive a plurality of strips connected by at least one flexible member. The flexible member is configured to permit the strips to pivot relative to each other such that the strips can shift between a flat configuration and an arcuate configuration. The tissue sample holder is rotatable to successively index each chamber to the cutter lumen such that tissue samples may be collected in the strips. The strips may be removed from the tissue sample holder and placed in a tissue sample holder container for imaging of tissue samples.

Leica Biosystems is a global leader in workflow solutions and automation, providing anatomic pathology labs and researchers a comprehensive product range for each step in the pathology process from sample preparation and staining to imaging and reporting. Leica Biosystems has published on their website informational booklets that are accessible via download and that contain information on various aspects of the pathology process. These booklets include, but are not limited to: “An Introduction to Tissue Processing” by Geoffrey Rolls, “101 Steps to Better Histology,” and “Total Histology Solutions,” all of which are available via www.leicabiosystems.com.

At several steps during tissue processing using conventional techniques and instruments, it may be necessary to manually manipulate the tissue. This manual manipulation may take time and introduce the possibility of human error causing mistakes during the processing of tissue. Any and all mistakes during the processing of tissue may make the pathological examination of the tissue much more problematic to achieve the desired goal of having an accurate diagnosis. Thus, it is understood that a desired goal of modern tissue processing is the reduction of the requirement that tissue be manually manipulated.

International Pat. Pub. No. WO 2013/192606, entitled “Biopsy Tissue Sample Transport Device and Method of Using Thereof,” published on Dec. 27, 2013, describes a biopsy tissue sample transport device and method of using the same including a tissue storage assembly having a sample container, having a holding structure to hold a tissue sample, the holding structure having a sample access opening formed in a sidewall; a housing that receives the tissue storage assembly, the housing comprising an assembly insertion opening through which the tissue storage assembly is inserted into the housing; a sealing member configured to engage and substantially seal the sample access opening of the holding structure of the sample container of the tissue storage assembly; and a lid to engage and substantially seal the assembly insertion opening of the housing.

International Pat. Pub. No. WO 2013/192607, entitled “Tissue Sample Container and Methods,” published on Dec. 27, 2013, describes a tissue sample container including a base having a plurality of sample holding sections, which are configured to receive a plurality of tissue samples in a given orientation and are demarcated by section walls; and a lid configured to sealingly engage the base. The sample holding sections are sized and shaped to correspond to a specific tissue sample size and shape such that the base in cooperation with the section walls, maintain the given orientation and identity of the tissue samples within respective sample holding sections.

International Pat. Pub. No. WO 2014/151603, entitled “Biopsy Device,” published on Sep. 25, 2014, describes a biopsy device that includes a probe, a holster, and a tissue sample holder for collecting tissue samples. The probe includes a needle and a hollow cutter. The tissue sample holder includes a housing having a plurality of chambers that are configured to receive a plurality of strips connected by at least one flexible member. The flexible member is configured to permit the strips to pivot relative to each other such that the strips can shift between a flat configuration and an arcuate configuration. The tissue sample holder is rotatable to successively index each chamber to the cutter lumen such that tissue samples may be collected in the strips. The strips may be removed from the tissue sample holder and placed in a tissue sample holder container for imaging of tissue samples.

U.S. Pat. No. 7,715,523, entitled “System and Apparatus for Rapid Stereotactic Breast Biopsy Analysis,” issued on May 11, 2010, and U.S. Pat. No. 8,503,602, entitled “System and Apparatus for Rapid Stereotactic Breast Biopsy Analysis,” issued on Aug. 6, 2013, both describe a stereotactic breast biopsy apparatus and system that may comprise an x-ray source, a digital imaging receptor, and a biopsy specimen cassette, wherein the x-ray source is provided with a means for displacing the beam axis of the x-ray source from a working biopsy corridor beam axis to permit an unobstructed illumination of the biopsy specimen and thereby produce biopsy x-ray images directly in the procedure room for immediate analysis. Some examples of the benefits may be, but are not limited to, a more rapid analysis of biopsy specimen digital images, post-processing image capability, and decreased procedure time and diminution of patient bleeding complications and needle discomfort.

U.S. Pat. No. 8,485,987, entitled “Tissue Handling System with Reduced Operator Exposure,” issued Jul. 16, 2016, describes a tissue handling system includes a biopsy device having an invasive unit with tissue-receiving and tissue-severing components being capable of harvesting and bringing at least one tissue sample to a point outside the body of a patient. The tissue handling system further includes a tissue collecting device adapted to be brought in detachable operative engagement with the tissue-receiving components of the biopsy device to remove the at least one tissue sample. Additionally, the tissue handling device comprises a tissue storage container configured to receive the at least one tissue sample, the entire tissue collecting device, or the part of the collecting device that contains the at least one tissue sample. The tissue storage container further is configured to receive a volume of preserving agent. The tissue handling system also comprises a vessel including the preserving agent adapted to be gas-tightly mated or coupled to the tissue storage container.

U.S. Pat. No. 8,802,034, entitled “Tissue Container for Molecular and Histology Diagnostics Incorporating a Breakable Membrane,” issued on Aug. 12, 2014, describes a container for storing a biological sample for molecular diagnostic testing and/or histological testing. The container includes a first chamber for receiving a sample holder therein, a second chamber, and a closure for enclosing the container. A breakable membrane, such as a pierce-able foil, extends within the container and separates the two chambers. When the breakable membrane is broken, fluid can pass between the first and second chambers. The membrane may be broken through an activator on the closure, such as a depressible member or a rotatable carrier, causing the sample holder to break through the membrane.

U.S. Pat. No. 9,056,317, entitled “Tissue Container for Molecular and Histology Diagnostics Incorporating a Breakable Membrane,” issued on Jun. 16, 2016 describes a container for storing a biological sample for molecular diagnostic testing and/or histological testing. The container includes a first chamber for receiving a sample holder therein, a second chamber, and a closure for enclosing the container. A breakable membrane, such as a pierce able foil, extends within the container and separates the two chambers. When the breakable membrane is broken, fluid can pass between the first and second chambers. The membrane may be broken through an activator on the closure, such as a depressible member or a rotatable carrier, causing the sample holder to break through the membrane.

While several systems and methods have been made and used for obtaining and processing a biopsy sample, it is believed that no one prior to the inventor has made or used the invention described in the appended claims.

### SUMMARY

The first aspect of the instant claimed invention is an apparatus comprising: (a) a base, wherein the base comprises: (i) a floor, (ii) a set of sidewalls, wherein the floor and the sidewalls cooperate to define a compartment, wherein the compartment is configured to receive a tissue sample tray, and (ii) a plurality of bosses extending upwardly from the floor within the compartment, wherein the bosses are configured to maintain positioning of a tissue sample tray received in the compartment; and (b) a lid configured to cooperate with the base to transition between an open position and a closed position, wherein the lid is configured to enable placement of a tissue sample tray in the compartment when the lid is in the open position, wherein the lid is configured to enclose a tissue sample tray in the compartment when the lid is in the closed position; and (c) a plurality of openings, wherein the plurality of openings are formed through one or both of the sidewalls or the lid, wherein the openings are configured to enable fluid to reach a tissue sample tray enclosed in the compartment by the lid.

The second aspect of the instant claimed invention is an apparatus comprising: (a) a tissue sample tray, wherein the tissue sample tray comprises: (i) a plurality of tissue sample strips, wherein each tissue sample strip defines a respective tissue receiving chamber, and (ii) a pull tab located proximal to the tissue sample strips, wherein the tissue sample tray is configured to flexibly transition between an arcuate configuration and a flattened configuration; and (b) a container, wherein the container comprises: (i) a base, wherein the base comprises: (A) a compartment, wherein the compartment is configured to receive the tissue sample strips, and (B) a tab mount, wherein the tab mount is configured to receive the pull tab, wherein the tab mount is located external to the compartment, and (ii) a lid, wherein the lid is configured to selectively enclose the tissue sample strips in the compartment.

The third aspect of the instant claimed invention is a method comprising: (a) obtaining a plurality of tissue samples from a patient via a biopsy device, wherein the biopsy device deposits the tissue samples on corresponding tissue sample strips of a tissue sample tray, wherein the tissue sample tray is in an arcuate configuration when the biopsy device deposits the tissue samples on the corresponding tissue sample strips of the tissue sample tray; (b) removing the tissue sample tray from the biopsy device, wherein the tissue samples remain disposed on the corresponding tissue sample strips during the removal of the tissue sample tray from the biopsy device; (c) placing the tissue sample tray in a container; (d) closing a lid of the container to thereby enclose the tissue samples and the corresponding tissue sample strips in the container, wherein the tissue sample tray is in a flattened configuration with the tissue samples and the corresponding tissue sample strips enclosed in the container; and (e) placing the container in a radiograph machine to obtain one or more radiographic images of the tissue samples, wherein the tissue samples remain disposed on the corresponding tissue sample strips during the act of removing the tissue sample tray from the biopsy device, during the act of placing the tissue sample tray in the container, during the act of closing the lid of the container, and during the act of placing the container in the radiograph machine, such that the tissue samples are not removed from the corresponding tissue sample strips during or between the performance of said acts.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary biopsy device;

FIG. 2 depicts an exploded perspective view of a tissue sample holder assembly of the biopsy device of FIG. 1;

FIG. 4 depicts a perspective view of the tissue sample tray of FIG. 3 in a flattened configuration;

FIG. 5A depicts a top perspective view of an exemplary container that is configured to receive and support the tissue sample tray of FIG. 3, with the container in a closed configuration;

FIG. 5B depicts a top perspective view of the container of FIG. 5A in an open configuration;

FIG. 5C depicts a top perspective view of the tissue sample tray of FIG. 3 positioned in the container of FIG. 5A, with the container in the open configuration;

FIG. 5D depicts a top perspective view of the tissue sample tray of FIG. 3 positioned in the container of FIG. 5A, with the container in the closed configuration;

FIG. 6 depicts a bottom perspective view of the container of FIG. 6, with the container in a closed configuration;

FIG. 7 depicts another bottom perspective view of the container of FIG. 6, with the container in a closed configuration;

FIG. 8 depicts a bottom perspective view of a lid of the container of FIG. 6;

FIG. 9 depicts a top perspective view of a base of the container of FIG. 6;

FIG. 10A depicts a perspective view of a portion of an exemplary radiograph machine with a drawer in an open position, and with the container of FIG. 6 positioned for insertion in the drawer;

FIG. 10B depicts a perspective view of the portion of the radiograph machine of FIG. 10A with the drawer in the open position, and with the container of FIG. 6 positioned in the drawer;

FIG. 10C depicts a perspective view of the portion of the radiograph machine of FIG. 10A with the drawer in a closed position; and

FIG. 11 depicts a perspective view of the container of FIG. 6 positioned in a cup of fixation fluid.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

The following “Parts List” giving the number and name of each part shown in the accompanying drawings is included to guide the reader:

| Reference | |
|---|---|
| Number | Part |
| 10 | biopsy device |
| 20 | probe assembly |
| 22 | needle |
| 24 | tip |
| 26 | lateral aperture |
| 30 | holster assembly |
| 40 | tissue sample holder assembly |
| 42 | outer cover |
| 44 | rotatable member |
| 46 | strip receiving chamber |
| 48 | plug receiving chamber |
| 50 | plug |
| 100 | tissue sample tray |
| 110 | tissue sample strip |
| 112 | strip sidewalls |
| 114 | floor |
| 116 | openings |
| 120 | tissue receiving chamber |
| 122 | distal opening |
| 130 | pull tab |
| 132 | tab opening |
| 140 | proximal panel |
| 142 | living hinge |
| 200 | container |
| 210 | base |
| 212 | base body |
| 214 | base openings |
| 216 | label panel |
| 218 | distal panel |
| 220 | tab mount |
| 222 | detent opening |
| 224 | pin opening |
| 226 | base sidewall |
| 227 | base floor |
| 228 | compartment |
| 230 | strip separation bosses |
| 232 | tray positioning bosses |
| 240 | container positioning guides |
| 242 | container orientation guide |
| 250 | container lid |
| 252 | lid body |
| 254 | first set of lid openings |
| 256 | second set of lid openings |
| 258 | tab |
| 260 | detent protrusion |
| 270 | engagement boss |
| 272 | edge |
| 280 | pin |
| 300 | radiograph machine |
| 310 | drawer |
| 320 | container receiving recess |
| 322 | drawer sidewall |
| 324 | container orientation recess |
| 400 | cup |
| 410 | fixation fluid |
| 420 | cup lid |

I. Exemplary Biopsy Device

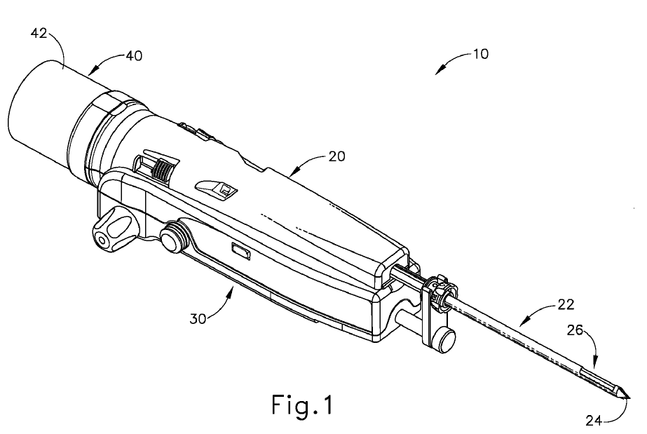

FIG. 1 depicts an exemplary biopsy device (10) that can be used to acquire tissue samples from a patient. Biopsy device (10) comprises a probe assembly (20), a holster assembly (30), and a tissue sample holder assembly (40). Probe assembly (20) includes a distally projecting needle (22) that has a tissue piercing tip (24) and a lateral aperture (26) that is located proximal to tip (24). A tubular cutter (not shown) is slidably disposed in needle (22) and is operable to sever tissue that is protruding through lateral aperture (26). The severed tissue samples are communicated proximally through the lumen of the cutter to tissue sample holder assembly (40), as described below. In some versions, probe assembly (20) is coupled with a control module that is operable to provide communication of vacuum, saline, and/or atmospheric air to probe assembly (20).

Holster assembly (30) includes features that are operable to drive the cutter, features that are operable to fire needle (22) distally into tissue, and features that are operable to rotate needle (22) about a longitudinal axis of needle (22). In some versions, holster assembly (30) is coupled with a control module via a cable that is operable to provide electrical power and/or other electrical signals to holster assembly (30). In addition, or in the alternative, holster assembly (30) may receive a pressurized medium (e.g., air, hydraulic fluid, etc.) in order to provide motive force to drive the cutter of probe assembly (20).

In the present example, probe assembly (20) and holster assembly (30) are configured for use in a stereotactic image guided biopsy procedure. By way of example only, probe assembly (20) and holster assembly (30) may be constructed and operable in accordance with at least some of the teachings of U.S. Pub. No. 2014/0039343, entitled “Biopsy System,” published Feb. 6, 2014, the disclosure of which is incorporated by reference herein. Alternatively, probe assembly (20) and holster assembly (30) may be configured for use in (or otherwise be used in) an ultrasound image guided biopsy procedure and/or an MRI guided biopsy procedure. By way of further example only, probe assembly (20) and holster assembly (30) may be constructed and operable in accordance with at least some of the teachings of U.S. Pub. No. 2013/0150751, entitled “Biopsy Device with Slide-In Probe,” published Jun. 13, 2013, the disclosure of which is incorporated by reference herein. Alternatively, probe assembly (20) and holster assembly (30) may be constructed and operable in any other suitable fashion.

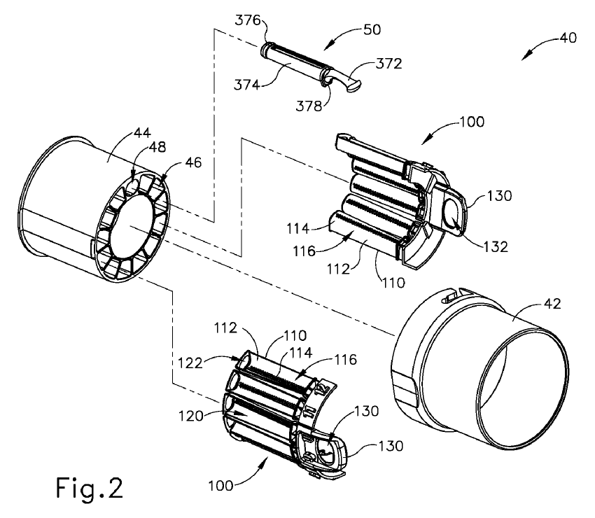

As noted above, tissue sample holder assembly (40) is configured to receive tissue samples that are severed by the cutter from tissue protruding through lateral aperture (26). As shown in FIG. 2, tissue sample holder assembly (40) of this example comprises a cylindraceous outer cover (42) that is removably coupled with probe assembly (20). A rotatable (44) member is rotatably positioned within cover (42). Rotatable member (44) defines an angularly spaced array of strip receiving chambers (46) and a plug chamber (48), such that chambers (46, 48) together an annular arrangement. Rotatable member (44) is rotatable relative to probe assembly (20) to selectively index chambers (46, 48) relative to the cutter. In some versions, drive components in holster assembly (30) drive rotation of rotatable member (44). In some other versions, rotatable member (44) is driven manually by the operator manually grasping some portion of tissue sample holder assembly (40).

As also shown in FIG. 2, tissue sample holder assembly (40) further includes a pair of tissue sample trays (100). Each tissue sample tray (100) comprises a set of distally projecting tissue sample strips (110). Each tissue sample strip (110) is configured for removable insertion into a corresponding strip receiving chamber (46) of rotatable member (44). Each tissue sample strip (110) comprises a set of strip sidewalls (112) joined by a floor (114). Strip sidewalls (112) and floor (114) cooperate to define a tissue receiving chamber (120), such that each tissue sample strip (110) is configured to receive a corresponding tissue sample. Floor (114) defines a plurality of openings (116) that are sized to provide communication of suction and fluids therethrough, while preventing communication of tissue samples therethrough. It should be understood that suction may be communicated through strip receiving chambers (46) to reach tissue receiving chambers (120) via openings (116). Each tissue sample strip (110) of the present example also includes a distal opening (122). Distal opening (122) is sized and configured to enable a severed tissue sample to pass therethrough in order for the tissue sample to be deposited into tissue receiving chamber (120).

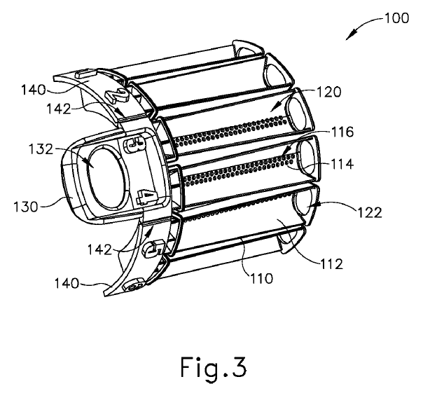

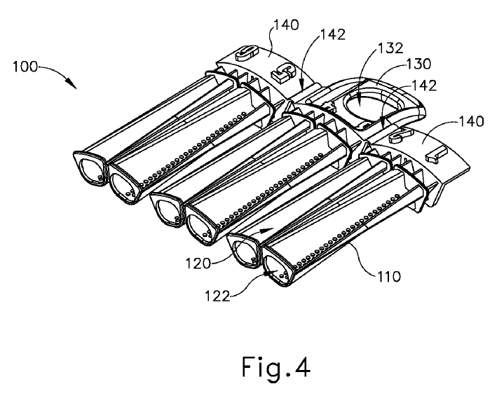

As best seen in FIGS. 3-4, each tissue sample tray (100) further includes a proximally projecting pull tab (130) that defines a tab opening (132). Pull tab (130) is configured to facilitate grasping of tissue sample tray (100) by an operator. Tissue sample tray (100) also includes a set of proximal panels (140). In the present example, two tissue sample strips (110) project distally relative to a corresponding panel (140) of the set of panels (140). Pull tab (130) projects proximally from the centrally positioned panel (140). Panels (140) are flexibly joined together by living hinges (142). Living hinges (142) enable tissue sample tray (100) to transition between the arcuate configuration shown in FIG. 3 and the flattened configuration shown in FIG. 4. In the arcuate configuration, tissue sample tray (100) is configured to fit in rotatable member (44). In the flattened configuration, tissue sample tray (100) is configured to fit in a container (200) as will be described in greater detail below.

As noted above, rotatable member (44) is rotatable relative to probe assembly (20) to selectively index strip receiving chambers (46) relative to the cutter, to thereby selectively index tissue receiving chambers (120) of tissue sample strips (110) relative to the cutter. Rotatable member (44) is also operable to index plug receiving chamber (48) relative to the cutter. When rotatable member (44) is angularly positioned to index plug receiving chamber (48) relative to the cutter, plug (50) may be removed from plug receiving chamber (48) to enable insertion of a biopsy site marker applier instrument (or some other kind of instrument) through the cutter and needle assembly (22), thereby providing an access path to the biopsy site via lateral aperture (26). Otherwise, plug (50) may be left in plug receiving chamber (48) during operation of biopsy device (10), thereby sealing plug receiving chamber (48).

By way of example only, tissue sample holder (40) may be configured and operable in accordance with at least some of the teachings of U.S. Pub. No. 2014/0039343, entitled “Biopsy System,” published Feb. 6, 2014, the disclosure of which is incorporated by reference herein and/or U.S. Pub. No. 2014/0275999, entitled “Biopsy Device,” published Sep. 18, 2014, the disclosure of which is incorporated by reference herein.

II.Exemplary Container to Support Tissue Sample Tray

As noted above, tissue sample tray (100) is flexible such that tissue sample tray (100) may readily transition between the arcuate configuration shown in FIG. 3 and the flattened configuration shown in FIG. 4. While this flexibility may be beneficial to enable an operator to selectively change the configuration of tissue sample tray (100) based on the needs at hand, this flexibility may also provide a need to provide structural support to tissue sample tray (100) in order to maintain the positioning and arrangement of tissue sample strips (110) based on how tissue sample tray (100) will be handled.

In addition, while the “U”-shaped profile provided by strip sidewalls (112) and floor (114) may enable an operator to easily pull tissue samples from each tissue receiving chamber (120) (i.e., via the opening defined between sidewalls (112)), it may be desirable to provide temporary enclosure of each tissue receiving chamber (120) to fully contain tissue samples in respective tissue receiving chambers (120), particularly when tissue sample tray (100) is in the flattened configuration shown in FIG. 4.

By way of example only, it may be desirable to provide the above-described additional structural support to tissue sample tray (100), as well as the enclosure of each tissue receiving chamber (120) to fully contain tissue samples in respective tissue receiving chambers (120), when the tissue samples are to be positioned in an imaging machine such as a radiograph machine. Similarly, it may be desirable to provide the above-described additional structural support to tissue sample tray (100), as well as the enclosure of each tissue receiving chamber (120) to fully contain tissue samples in respective tissue receiving chambers (120), when the tissue samples are to be contained in a fixation fluid (e.g., formalin).

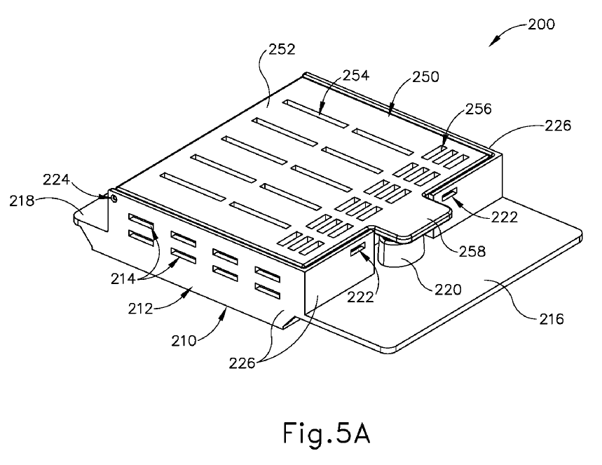

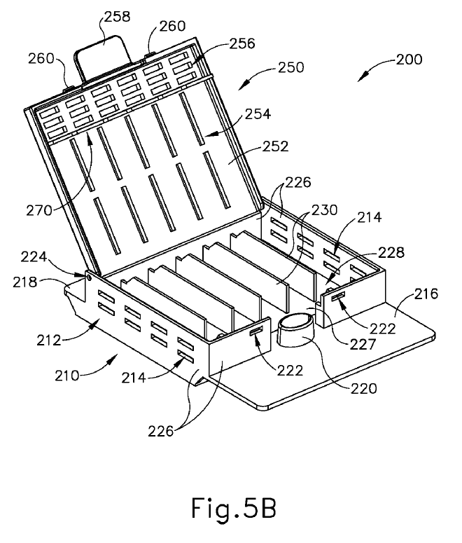

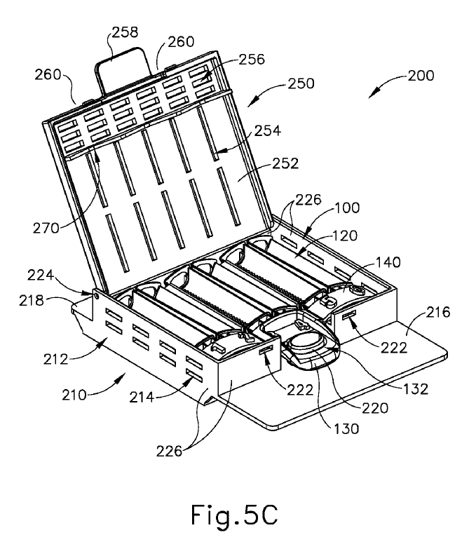

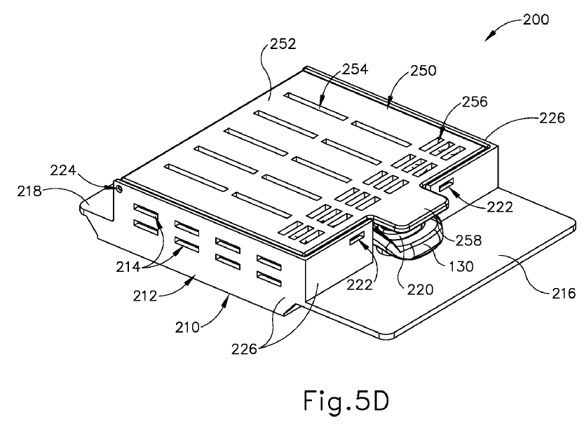

FIGS. 5A-7 show an exemplary container (200) that is operable to provide structural support to tissue sample tray (100), as well as the enclosure of each tissue receiving chamber (120) to fully contain tissue samples in respective tissue receiving chambers (120). Container (200) of this example comprises a base (210) and a container lid (250), which is pivotably coupled with base (210). As best seen in FIGS. 5B and 9, base (210) of the present example comprises a rigid base body (212) having a set of base sidewalls (226), a base floor (227), a proximally projecting label panel (216), and a distal panel (218). A plurality of elongate base openings (214) and a pair of pin openings (224) are formed in a corresponding pair of base sidewalls (226). A pair of detent openings (222) are formed through another corresponding pair of base sidewalls (226). Base sidewalls (226) and base floor (227) all cooperate to define a base compartment (228), which is configured to receive tissue sample tray (100) as described below.

Label panel (216) is configured to receive information associated with the tissue samples that are contained within container (200) as will be described in greater detail below. By way of example only, label panel (216) may be written on with a marking pen. By way of further example only, a sticker or other kind of label may be applied to label panel (216). Various other suitable ways in which information may be provided on label panel (216) will be apparent to those of ordinary skill in the art in view of the teachings herein.

A tab mount (220) projects upwardly from label panel (216). Tab mount (220) is sized and configured for insertion in tab opening (132) of tissue sample tray (100), as will be described in greater detail below. As best seen in FIGS. 5B and 9, base (210) of the present example further includes a set of strip separation bosses (230) and a set of tray positioning bosses (232). Bosses (230, 232) extend upwardly from base floor (227). Bosses (230, 232) are configured to engage tissue sample tray (100) to thereby guide and maintain positioning of tissue sample tray (100) in base compartment (228) of container (200), as will also be described in greater detail below. It should also be understood that tray positioning bosses (232) may be configured to prevent an operator from mistakenly inserting tissue sample tray (100) upside-down in base compartment (228) (i.e., such that floors (114) of tissue sample strips (110) face container lid (250) rather than facing base floor (227)).

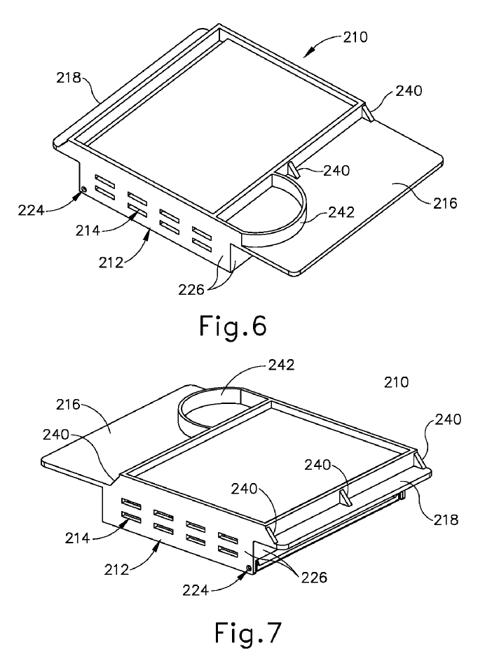

As best seen in FIGS. 6-7, the underside of base (210) includes a set of container positioning guides (240) and a container orientation guide (242). In particular, three container positioning guides (240) are positioned under distal panel (218) while two container positioning guides (240) are positioned under label panel (216). Container positioning guides (240) are in the form of rigid, obliquely angled ribs in the present example. Other suitable configurations that may be used for container positioning guides (240) will be apparent to those of ordinary skill in the art in view of the teachings herein. Container orientation guide (242) is in the form of a rigid, arcuate rib positioned under label panel (216) in this example. Again, other suitable configurations that may be used for container orientation guide (242) will be apparent to those of ordinary skill in the art in view of the teachings herein. As will be described in greater detail below, guides (240, 242) are configured to engage structures in a drawer (310) of a radiograph machine (300) to thereby provide consistently proper orientation and positioning of container (200) in drawer (310).

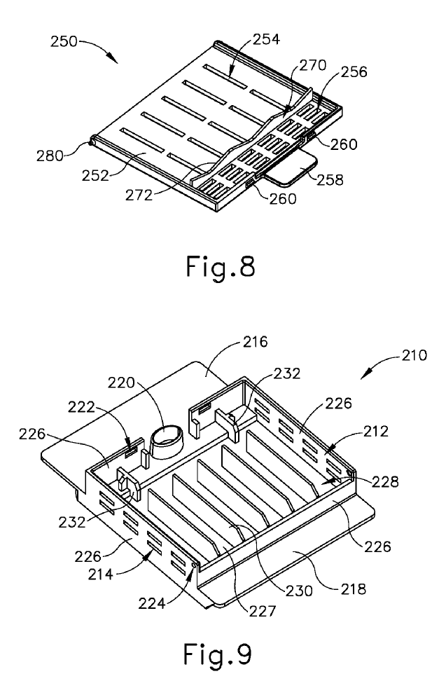

As best seen in FIGS. 5B and 8, container lid (250) of the present example includes a rigid lid body (252) that defines a first set of lid openings (254) and a second set of lid openings (256). A tab (258) and a pair of detent protrusions (260) project proximally from lid body (252). An engagement boss (270) is located on the underside of lid body (252) and defines an edge (272) having a profile similar to a triangular waveform. First set of lid openings (254) are positioned on one side of engagement boss (270) while a second set of lid openings (256) are positioned on the other side of engagement boss (270). In the present example, first set of lid openings (254) are oriented perpendicularly relative to second set of lid openings (256), though it should be understood that any other suitable orientations and relationships may be used. A pair of pins (280) extend laterally outwardly from lid body (252). Pins (280) are disposed in pin openings (224) of base (210), thereby providing a pivotal coupling between container lid (250) and base (210). Container lid (250) is thus configured to pivot between the closed position shown in FIG. 5A and the open position shown in FIG. 5B.

Detent protrusions (260) are positioned and configured to engage corresponding detent openings (222) of base (210), to thereby selectively maintain container lid (250) in the closed position shown in FIG. 5B. In particular, detent protrusions (260) and detent openings (222) cooperate to prevent container lid (250) from being inadvertently opened; yet detent protrusions (260) and detent openings (222) still enable an operator to intentionally open container lid (250).

As shown in FIG. 5C, an operator may position a tissue sample tray (100), with tissue samples disposed in one or more corresponding tissue receiving chambers (120), in base compartment (228). Base (210) is thus configured to receive tissue sample tray (100) in base compartment (228) with tissue sample tray (100) in the flattened configuration. In the present example, strip separation bosses (230) are in the form of straight ribs extending perpendicularly upwardly from base floor (227). Strip separation bosses (230) are parallel with each other. Strip separation bosses (230) are configured to fit between tissue sample strips (110). Since strip separation bosses (230) are rigid while tissue sample strips (110) are flexible, strip separation bosses (230) are configured to provide structural support that maintains parallel relationships between tissue sample strips (110). As the operator inserts tissue sample tray (100) into base compartment (228), tray positioning bosses (232) assist in guiding tissue sample tray (100) into proper positioning relative to base (210), thereby facilitating positioning of strip separation bosses (230) between tissue sample strips (110). In addition, as noted above, tab mount (220) is received in tab opening (132) of pull tab (130) when tray (100) is inserted in base (210). It should therefore be understood that a portion of pull tab (130) protrudes from base compartment (228) while tissue sample strips (110) and proximal panels (140) of tissue sample tray (100) are positioned in base compartment (228). In some versions, there is an interference fit between tab mount (220) and tab opening (132), such that tissue sample tray (100) will be substantially held in place in base (210) by friction even while container lid (250) is in the open position.

As shown in FIG. 5D, the operator may close container lid (250) after tissue sample tray (100) is properly positioned in base compartment (228). As container lid (250) is closed, edge (272) of engagement boss (270) engages tissue sample strips (110) to further flatten tissue sample tray (100) and/or to further secure the positioning of tissue sample strips (110) within container (200). In addition, container lid (250) provides a substantially close fit relative to the upper edges of strip sidewalls (112), such that container lid (250) will prevent tissue samples from exiting tissue receiving chambers (120) when container lid (250) is in the closed position. As can also be seen in FIG. 5D, tab (258) of container lid (250) is positioned over tab mount (258) of base (210) and pull tab (130) of tissue sample tray (100) when container lid (250) is in the closed position. Tab (258) may thus prevent pull tab (130) from being inadvertently pulled off of tab mount (258). It should also be understood that tab (258) may be grasped or otherwise manipulated by the operator to facilitate transitioning of container lid (250) between the open and closed positions.

As described in greater detail below, openings (214, 254, 256) of container (200) are configured to permit the ingress of fixation fluid into container (200) while container lid (250) is in a closed configuration, thereby allowing the fixation fluid to reach tissue in tissue receiving chambers (120) that are contained in container (200). However, before container (200) is placed in a fixation fluid, it should be understood that tissue sample tray (100) may be leaking some fluid from the biopsy procedure (e.g., blood, saline, etc.). It should also be understood that it may be desirable to contain these leaked fluids in base (210), to prevent or otherwise minimize the further leakage of such fluids from container (200) to work surfaces or other equipment (e.g., radiograph machine (300)). To that end, base body (212) is configured to contain at least some such fluids in base compartment (228) while container (200) is kept in a parallel relationship with the ground (i.e., with the underside of base (210) facing the ground). In other words, sidewalls (226) and floor (227) cooperate to a fluid reservoir in the region of base compartment (228) that is beneath the lowermost base openings (214), such that fluids below the lowermost base openings (214) will stay in base compartment (228) so long as container (200) is kept in a parallel relationship with the ground (i.e., with the underside of base (210) facing the ground). Other suitable features that may be used to manage fluid leaking from tissue sample tray (100) will be apparent to those of ordinary skill in the art in view of the teachings herein.

III. Exemplary Processing of Tissue on Tissue Sample Tray in Container

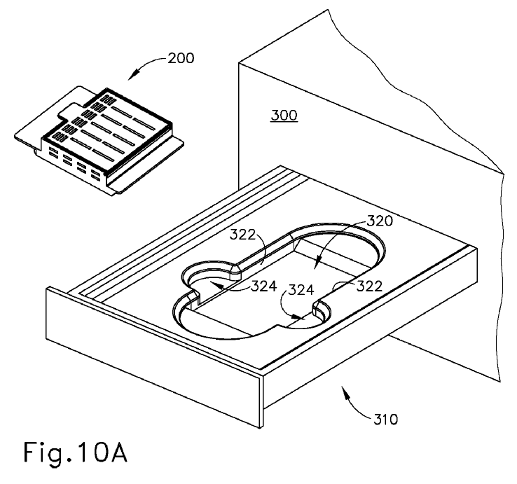

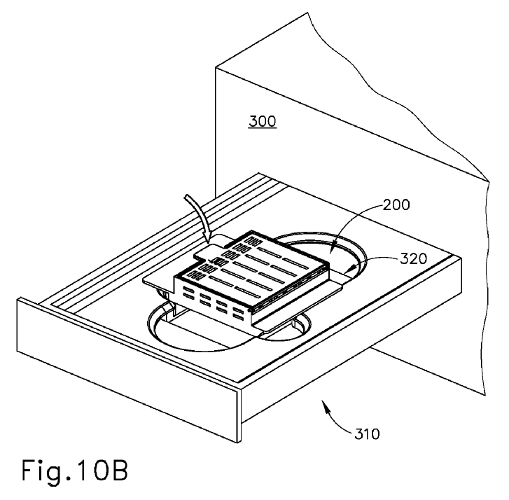

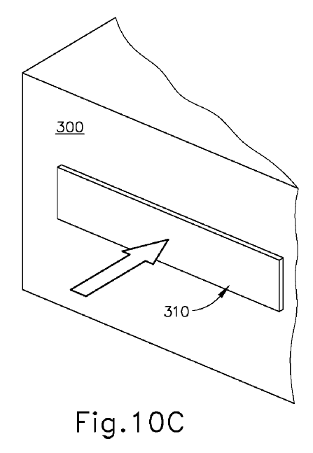

After tissue sample tray (100) is enclosed in container (200), with tissue samples thereby contained in respective tissue receiving chambers (120) of tissue sample tray (100), it may be desirable to obtain radiographic images of the tissue samples and then immerse the tissue samples in a fixation fluid. FIGS. 10A-10C show an exemplary radiograph machine (300) that may be used to obtain radiographic images of the tissue samples while the tissue samples are contained in respective tissue receiving chambers (120) of tissue sample tray (100), enclosed in container (200). Radiograph machine (300) of this example includes a drawer (310) that is operable to transition between an open position (FIGS. 10A-10B) and a closed position (FIG. 10C). When drawer (310) is in the open position, drawer (310) may removably receive container (200) with tissue sample tray (100). When drawer (310) is in the closed position, radiograph machine may be operated to obtain radiographic images of the tissue samples that are contained in respective tissue receiving chambers (120) of tissue sample tray (100), enclosed in container (200).

Drawer (310) of the present example defines a container receiving recess (320), which is configured to receive container (200). Container receiving recess (320) is defined in part by drawer sidewalls (322) and container orientation recesses (324). Drawer sidewalls (322) are substantially parallel to each other. Container orientation recesses (324) each have an arcuate profile, and are formed at the distal ends of respective, oppositely facing drawer sidewalls (322). It should be understood that container receiving recesses (320) may have any other suitable alternative configuration.

It should be understood that it may be desirable to ensure consistent, proper placement of container (200) in container receiving recess (320), in order to ensure accurate analysis of tissue samples by radiograph machine (300). To that end, container orientation guide (242) is configured to engage container orientation recess (324); and container positioning guides (240) are configured to engage drawer sidewalls (322). In particular, as the operator positions container (200) in container receiving recess (320) as shown in FIG. 10B, the complementary structures of container orientation guide (242) and container orientation recess (324) will ensure that container (200) is placed in container receiving recess (320) at the proper orientation. It should be understood that if the operator attempts to insert container (200) in container receiving recess (320) at an improper orientation, container orientation guide (242) will clearly prevent container (200) from achieving proper seating in container receiving recess (320). Similarly, as the operator positions container (200) in container receiving recess (320), container positioning guides (240) will engage the upper edges of drawer sidewalls (322) guide container (200) into a centered position within container receiving recess (320). In particular, the oblique orientation of container positioning guides (240) will provide a camming action against the upper edges of drawer sidewalls (322) guide container (200) into a centered position within container receiving recess (320).

In the present example, even with container (200) properly seated in container receiving recess (320), an upper portion of container (200) (including panels (216, 218), container lid (250), and an upper portion of base body (212)) will still be positioned above container receiving recess (320). In some other versions, additional portions of container (200) (or even the entirety of container (200)) may be configured to fit in container receiving recess (320).

Once container (200) is properly seated in container receiving recess (320), drawer (310) may be closed as shown in FIG. 10C. Radiograph machine (300) may then be activated to obtain one or more radiographic images of the tissue samples that are contained in respective tissue receiving chambers (120) of tissue sample tray (100), enclosed in container (200) in container receiving recess (320). It should be understood that the one or more radiographic images may be captured using X-rays. It should also be understood that radiograph machine (300) may be further configured to provide various kinds of tissue analysis or functionality, based on the acquired radiographic images and/or based on other data points (e.g., automatically highlight potential calcifications, and/or other anomalies, etc.). By way of example only, radiograph machine (300) may comprise a CoreVision® Specimen Radiography System manufactured by Faxitron Bioptics, LLC of Tucson, Ariz. Alternatively, any other suitable kind of radiograph machine (300) may be used as will be apparent to those of ordinary skill in the art in view of the teachings herein.

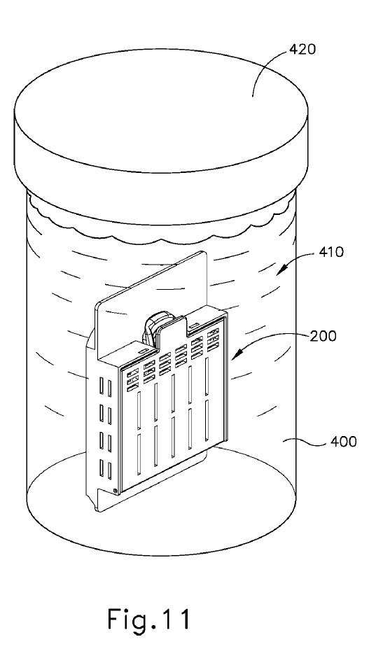

After the desired radiographic images are captured, it may be desirable to place the tissue samples in a fixation fluid (e.g., formalin, etc.). It may also be desirable to keep the tissue samples contained on tissue sample tray (100) in container (200) while the tissue samples are in the fixation fluid. To that end, the operator may open drawer (310) of radiograph machine (300) and retrieve container (200) from container receiving recess (320). The operator may then place container (200), without first opening container lid (250) or manipulating tissue samples held on tissue sample tray (100), into a cup (400) as shown in FIG. 11. In some instances, cup (400) may be pre-filled with a fixation fluid (410), such that container (200) is immediately immersed in fixation fluid (410). In some other instances, fixation fluid (410) may be introduced to cup after container (200) is first placed in cup (400). In either case, it should be understood that fixation fluid (410) may immediately pass into the interior of container (200) via openings (214, 254, 256). Fixation fluid (410) may thereby readily reach and immerse the tissue samples contained on tissue sample tray (100) in container (200). With container (200) and fixation fluid (410) in cup (400), the operator may then secure cup lid (420) to cup (400), thereby sealing container (200) and fixation fluid (410) in cup (400). After container (200) and fixation fluid (410) are sealed in cup (400), cup (400) may then be transported to another location for further processing, be set aside for storage, or be otherwise handled.

It should be understood from the foregoing that the combination of tissue sample tray (100) and container (200) will enable an operator to pull tissue samples directly from biopsy device (10), perform radiographic imaging of those tissue samples, and provide fixation of those tissue samples, without ever having to directly handle any of the tissue samples. In other words, the tissue samples may remain disposed in respective tissue receiving chambers (120) of tissue sample tray (100) during the entire process, from the time the tissue samples are initially deposited into respective tissue receiving chambers (120) during the biopsy procedure until the tissue samples are immersed in fixation fluid (410). By eliminating the need for separate, human manipulation of the tissue samples during this process, the combination of tissue sample tray (100) and container (200) may eliminate risks of mishandling that might otherwise occur in settings where tissue samples are individually manipulated by a human operator before radiographic imaging and/or before immersion in fixation fluid (410).

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An apparatus comprising:
(a) a base, wherein the base comprises:
(i) a floor,
(ii) a set of sidewalls, wherein the floor and the sidewalls cooperate to define a compartment, wherein the compartment is configured to receive a tissue sample tray, and
(ii) a plurality of bosses extending upwardly from the floor within the compartment, wherein the bosses are configured to maintain positioning of a tissue sample tray received in the compartment; and
(b) a lid configured to cooperate with the base to transition between an open position and a closed position, wherein the lid is configured to enable placement of a tissue sample tray in the compartment when the lid is in the open position, wherein the lid is configured to enclose a tissue sample tray in the compartment when the lid is in the closed position; and
(c) a plurality of openings, wherein the plurality of openings are formed through one or both of the sidewalls or the lid, wherein the openings are configured to enable fluid to reach a tissue sample tray enclosed in the compartment by the lid.

2. The apparatus of claim 1, wherein the base further comprises a tab mount, wherein the tab mount is configured to receive a pull tab of a tissue sample tray disposed in the compartment.

3. The apparatus of claim 2, wherein the tab mount is configured to provide an interference fit with a pull tab of a tissue sample tray disposed in the compartment.

4. The apparatus of claim 2, wherein the tab mount is located external to the compartment.

5. The apparatus of claim 2, wherein the base further comprises a label panel, wherein the tab mount is located on the label panel.

6. The apparatus of claim 1, wherein the base further comprises a label panel, wherein the label panel is located external to the compartment.

7. The apparatus of claim 1, wherein the lid is pivotably coupled with the base such that the lid is pivotable relative to the base to transition between the open position and the closed position.

8. The apparatus of claim 1, wherein the bosses include a first plurality of bosses, wherein the first plurality of bosses are configured to fit between tissue sample strips of a tissue sample tray received in the compartment.

9. The apparatus of claim 8, wherein the bosses of the first plurality of bosses comprise straight ribs extending perpendicularly upwardly from the floor.

10. The apparatus of claim 8, wherein the bosses of the first plurality of bosses are parallel with each other.

11. The apparatus of claim 1, wherein the bosses further include a second plurality of bosses, wherein the second plurality of bosses are configured to prevent insertion of a tissue sample tray into the compartment with the tissue sample tray in upside-down orientation.

12. The apparatus of claim 1, wherein the base has an underside that further comprises a plurality of guides, wherein the guides include a set of positioning guides configured to provide positioning of the base relative to a recess of a drawer of a radiograph machine.

13. The apparatus of 12, wherein the guides further include an orientation guide configured to restrict orientation of the base relative to a recess of a drawer of a radiograph machine.

14. The apparatus of claim 1, wherein the lid further comprises an engagement boss, wherein the engagement boss is configured to secure positioning of a tissue sample tray enclosed in the compartment by the lid.

15. The apparatus of claim 14, wherein the engagement boss is further configured to flatten a tissue sample tray enclosed in the compartment by the lid.

16. The apparatus of claim 1, wherein the plurality of openings comprises:
(i) a first set of openings formed through the sidewalls,
(ii) a second set of openings formed through the lid, and
(iii) a third set of openings formed through the lid, wherein the third set of openings is oriented perpendicularly relative to the second set of openings.

17. An apparatus comprising:
(a) a tissue sample tray, wherein the tissue sample tray comprises:
(i) a plurality of tissue sample strips, wherein each tissue sample strip defines a respective tissue receiving chamber, and
(ii) a pull tab located proximal to the tissue sample strips,
wherein the tissue sample tray is configured to flexibly transition between an arcuate configuration and a flattened configuration; and
(b) a container, wherein the container comprises:
(i) a base, wherein the base comprises:
(A) a compartment, wherein the compartment is configured to receive the tissue sample strips, and
(B) a tab mount, wherein the tab mount is configured to receive the pull tab, wherein the tab mount is located external to the compartment, and
(ii) a lid, wherein the lid is configured to selectively enclose the tissue sample strips in the compartment.

18. A method comprising:
(a) obtaining a plurality of tissue samples from a patient via a biopsy device, wherein the biopsy device deposits the tissue samples on corresponding tissue sample strips of a tissue sample tray, wherein the tissue sample tray is in an arcuate configuration when the biopsy device deposits the tissue samples on the corresponding tissue sample strips of the tissue sample tray;
(b) removing the tissue sample tray from the biopsy device, wherein the tissue samples remain disposed on the corresponding tissue sample strips during the removal of the tissue sample tray from the biopsy device;
(c) placing the tissue sample tray in a container;
(d) closing a lid of the container to thereby enclose the tissue samples and the corresponding tissue sample strips in the container, wherein the tissue sample tray is in a flattened configuration with the tissue samples and the corresponding tissue sample strips enclosed in the container; and
(e) placing the container in a radiograph machine to obtain one or more radiographic images of the tissue samples,
wherein the tissue samples remain disposed on the corresponding tissue sample strips during the act of removing the tissue sample tray from the biopsy device, during the act of placing the tissue sample tray in the container, during the act of closing the lid of the container, and during the act of placing the container in the radiograph machine, such that the tissue samples are not removed from the corresponding tissue sample strips during or between the performance of said acts.

19. The method of claim 18, further comprising:
(a) removing the container from the radiograph machine; and
(b) immersing the container in a fixation fluid, wherein the container has openings allowing the fixation fluid to immerse the tissue samples in the fixation fluid;
wherein the tissue samples continue to remain disposed on the corresponding tissue sample strips during the act of removing the container from the radiograph machine and during the act of immersing the container in the fixation fluid, such that the tissue samples are not removed from the corresponding tissue sample strips during or between the performance of said acts.

20. The method of claim 19, wherein the act of immersing the container in a fixation fluid comprises:
(i) depositing the container in a cup, wherein the cup further contains the fixation fluid, wherein the fixation fluid comprises formalin, and
(ii) securing a lid to the cup to thereby seal the container in the cup with the formalin.
